SEQUENCE LISTING (11 sequences)

SEQ ID NO: 1 - protein, 405 aa, Homo sapiens
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 2 - protein, 405 aa, Artificial Sequence
  Synthetic polypeptide
MQEKDASSQGFLPHFQHFATQAIHVGQDPEQWTSRAVVPPISLSTTFKQGAPGQHSGFTYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

SEQ ID NO: 3 - protein, 405 aa, Artificial Sequence
  Synthetic polypeptide
MQEKEASSQGFLPHFQHFATQAIHVGQEPEQWTSRAVVPPISPSVTFKQGAPGQHSGFTYSRSGNPTRNCLEKAVAALDGAKYCLAFASGLAATVTITHLLKAGDQIICMDDVYAGTNRYFRQVASEFGLKISFVDCSKIKLLEAAITPETKLVWIETPTNPTQKMTDIEACAHIVHKHGDIILVVDNTFMSPYFQRPLALGADICMCSATKYMNGHSDVVMGLVSVNCESLYNRLRFLQNSLGAVPSPIDCYLCNRGLKTLQVRMEKHFKNGMAVAQFLESNPWVEKVIYPGLPSHPQHELVKRQCTGCTGMVTFYIKGTLQHAEIFLKNLKLFTLAVSLGGFESLVELPAVMTHASVLKKDRDVLGISDTLIRLSVGLEDEEDLLEDLDQALKAAHPPSGSHS

SEQ ID NO: 4 - protein, 405 aa, Artificial Sequence
  Synthetic polypeptide
MQEKDASSQGFLPHFQHFATQAIHVGQEPEQWTSRAVVPLISLSTTFKQAAPGQHSGFTYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPVLKMIDIEACAHIVHKRGDIILVVDNTFMSPYFQRPLALGADICMCSATKYMNGHSDVVMGLVSVNCERLHNRLRFLQNSLGAVPSPLDCYLCNRGLKTLHVRMEKHFKNGMAVAQFLESNPGVEKVIYPGLPSHPQHELAKRQCTGCTGMITFYIKGTLQHAEIFLKNLKLFTLAVSLGGFESLVELPAIMTHASVPKNDRDVLGISDTLIRLSVGLEDEKDLLEDLDQALKAAHPPSGSHN

SEQ ID NO: 5 - protein, 405 aa, Artificial Sequence
  Synthetic polypeptide
MQEKDASSQGFLPHFQHFATQAIHVGQDPEQWTSRALVPPISLSTTFKQGAPGQHSGFTYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

SEQ ID NO: 6 - protein, 405 aa, Artificial Sequence
  Synthetic polypeptide
MQEKDASSQGFLPHFQHFATQAIHVGQDPEQWTSKALVPPISLSTTFKQGAPGQHSGFTYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVIGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

SEQ ID NO: 7 - protein, 405 aa, Pongo abelii
MQEKEASSQGFLPHFQHFATQAIHVGQEPEQWTSRAVVPPISPSVTFKQGAPGQHSGFVYSRSGNPTRNCLEKAVAALDGAKYCLAFASGLAATVTITHLLKAGDQIICMDDVYAGTNRYFRQVASEFGLKISFVDCSKIKLLEAAITPETKLVWIETPTNPTQKMTDIEACAHIVHKHGDIILVVDNTFMSPYFQRPLALGADICMCSATKYMNGHSDVVMGLVSVNCESLYNRLRFLQNSLGAVPSPIDCYLCNRGLKTLQVRMEKHFKNGMAVAQFLESNPWVEKVIYPGLPSHPQHELVKRQCTGCTGMVTFYIKGTLQHAEIFLKNLKLFTLAESLGGFESLVELPAVMTHASVLKKDRDVLGISDTLIRLSVGLEDEEDLLEDLDQALKAAHPPSGSHS

SEQ ID NO: 8 - protein, 405 aa, Macaca fascicularis
MQEKDASSQGFLPHFQHFATQAIHVGQEPEQWTSRAVVPLISLSTTFKQAAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPVLKMIDIEACAHIVHKRGDIILVVDNTFMSPYFQRPLALGADICMCSATKYMNGHSDVVMGLVSVNCERLHNRLRFLQNSLGAVPSPLDCYLCNRGLKTLHVRMEKHFKNGMAVAQFLESNPGVEKVIYPGLPSHPQHELAKRQCTGCTGMITFYIKGTLQHAEIFLKNLKLFTLAESLGGFESLVELPAIMTHASVPKNDRDVLGISDTLIRLSVGLEDEKDLLEDLDQALKAAHPPSGSHN

SEQ ID NO: 9 - protein, 405 aa, Pan troglodytes
MQEKDASSQGFLPHFQHFATQAIHVGQDPEQWTSRALVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 10 - protein, 405 aa, Pan paniscus
MQEKDASSQGFLPHFQHFATQAIHVGQDPEQWTSKALVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEACAHIVHKHGDIILVVDNTFMSPYFQRPLALGADICMYSATKYMNGHSDVVIGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 11 - protein, 415 aa, Artificial Sequence
  Synthetic polypeptide
MGGHHHHHHGGQEKDASSQGFLPHFQHFATQAIHVGQDPEQWTSRAVVPPISLSTTFKQGAPGQHSGFTYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAVSLGGFESLAELPAIMTHASVLKNDRDVLGISDTLIRLSVGLEDEEDLLEDLDQALKAAHPPSGSHS

(19) United States
(12) Patent Application Publication
(10) Pub. No.: US 2018/0008681 A1
(43) Pub. Date: Jan. 11, 2018

(54) HUMAN-ENZYME MEDIATED DEPLETION OF CYSTINE FOR TREATING PATIENTS WITH CYSTINURIA

(71) Applicant: Board of Regents, The University of Texas System, Austin, TX (US)

(72) Inventor: Everett STONE, Austin, TX (US)

(21) Appl. No.: 15/643,436

(22) Filed: Jul. 6, 2017

Related U.S. Application Data
(60) Provisional application No. 62/359,018, filed on Jul. 6, 2016.

(51) Int. Cl. A61K 38/51

(52) U.S. Cl. CPC A61K 38/51; A61K 47/60

(57) ABSTRACT

Methods and compositions related to the engineering of a protein with L-cyst(e)ine degrading enzyme activity are described. For example, in certain aspects there may be disclosed a modified cystathionine-γ-lyase comprising one or more amino acid substitutions and capable of degrading L-cyst(e)ine. Furthermore, certain aspects of the invention provide compositions and methods for the treatment of cancer with L-cyst(e)ine using the disclosed proteins or nucleic acids.

The present application claims the priority benefit of U.S. provisional application No. 62/359,018, filed Jul. 6, 2016, the entire contents of which is incorporated herein by reference.

### PARTIES TO JOINT RESEARCH AGREEMENT

The inventions disclosed and claimed herein were developed within the scope of a Joint Research Agreement between Aeglea BioTherapeutics, Inc. and The Board of Regents of The University of Texas System.

### BACKGROUND OF THE INVENTION

### 1. Field of the Invention

The present invention relates generally to the fields of medicine and biology. More particularly, it concerns the engineering of a primate enzyme with high cysteine/cystine degrading activity and stability suitable for human therapy. Even more particularly, it concerns compositions and methods for the treatment of cystinuria with enzymes that deplete both L-cystine and L-cysteine.

### 2. Description of Related Art

Cystinuria is a hereditary disorder caused by mutations in the SLC3A1 and SLC7A9 genes encoding the kidney proximal tubule's cystine and dibasic amino acid transporter that leads to abnormal excretion of cystine (the disulfide form of the amino acid cysteine) and the formation of cystine crystals/stones in the urinary tract. There are few therapeutics available to patients suffering from the hereditary disorder cystinuria wherein a defective kidney transporter is unable to re-uptake cystine during renal filtration. Cystine, the disulfide form of the amino acid L-cysteine, is highly insoluble and in cystinuria patients reaches high concentrations in the urinary tract resulting in the formation of cystine crystals and stones. Existing therapies that reduce circulating cystine levels partially prevent urinary tract stone formation but have significant adverse effects that limit their use.

### SUMMARY OF THE INVENTION

The present invention concerns methods of utilizing an engineered human cystathionine-gamma-lyase enzyme that efficiently converts cystine to cysteine-persulfide, which subsequently decays to free cysteine and H₂S, such that that it is a suitable therapy for treating cystinuria patients by preventing cystine accumulation and formation of stones in the kidney and urinary tract. As cystine is a non-essential amino acid that is normally produced by most cells, no toxicities have been found to be induced by long-term cystine depletion in animal models. Likewise, as this enzyme is comprised of a human sequence it is not likely to induce adverse immunological responses. The ability of a cystine degrading therapeutic to non-toxically ablate the total levels of circulating cystine indicate that it would be a superior embodiment for preventing cystine stone formation than existing therapeutic regimens.

The present invention concerns the engineering of primate cystathionine-gamma-lyase (CGL) enzymes such that both L-cystine and L-cysteine (referred to herein as L-cyst(e)ine) can be efficiently degraded from serum, and providing the modified CGL enzymes in a formulation suitable for human cancer therapy. To develop an enzyme displaying low K_{M} and high catalytic activity, k_{cat}, as compared to the native enzyme, the inventors engineered the native enzyme by modifying selected amino acids, which modifications result in an enzyme having dramatically improved enzymatic properties. As such, CGL enzymes modified as described herein overcome a major deficiency in the art by providing novel enzymes that comprise human or primate polypeptide sequences having L-cyst(e)ine-degrading catalytic activity as compared to the native enzyme. As such, these modified enzymes may be suitable for cancer therapy and have low immunogenicity and improved serum stability.

Accordingly, in one embodiment there is provided a modified polypeptide, particularly an enzyme variant with L-cyst(e)ine degrading activity derived from primate enzymes related to cystathionine-γ-lyase (CGL) enzymes. For example, an enzyme variant may have an amino acid sequence selected from the group consisting of SEQ ID NOs: 2-6. For example, the variant may be derived from a human enzyme, such as human CGL. In certain aspects, there may be a polypeptide comprising a modified primate CGL capable of degrading L-cyst(e)ine. In some embodiments, the polypeptide may be capable of degrading L-cyst(e)ine under physiological conditions. For example, the polypeptide may have a catalytic efficiency for L-cyst(e)ine (k_{cat}/K_{M}) of at least or about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10⁴, 10⁵, 10⁶ s⁻¹ M⁻¹ or any range derivable therein.

An unmodified polypeptide may be a native CGL, particularly a human isoform or other primate isoform. For example, the native human CGL may have the sequence of SEQ ID NO: 1. Non-limiting examples of other native primate CGL include Pongo abelii CGL (Genbank ID: NP_001124635.1; SEQ ID NO: 7), Macaca fascicularis CGL (Genbank ID: AAW71993.1; SEQ ID NO: 8), Pan troglodytes CGL (Genbank ID: XP_513486.2; SEQ ID NO: 9), and Pan paniscus CGL (Genbank ID: XP_003830652.1; SEQ ID NO: 10). Exemplary native polypeptides include a sequence having about, at least 60%, 65%, 70%, 75%, 80%, 85%, 90%, 95%, 96%, 97%, 98%, 99% or 100% identity (or any range derivable therein) to SEQ ID NOs: 1 or 7-10 or a fragment thereof. For example, the native polypeptide may comprise at least or up to about 10, 20, 30, 40, 50, 60, 70, 80, 90, 100, 150, 200, 250, 300, 350, 400, 405 residues (or any range derivable therein) of the sequence of SEQ ID NOs: 1 or 7-10.

In some embodiments, the native CGL may be modified by one or more other modifications, such as chemical modifications, substitutions, insertions, deletions, and/or truncations. In a particular embodiment, the native CGL may be modified by substitutions. For example, the number of substitutions may be one, two, three, four or more. In further embodiments, the native CGL may be modified in the substrate recognition site or any location that may affect substrate specificity. For example, the modified polypeptide may have the at least one amino acid substitution at an amino acid position corresponding to amino acid position 59 and/or 339 of SEQ ID NOs: 1 or 7-10. In these examples, the first methionine of each sequence corresponds to amino acid position 1, and each amino acid is numbered sequentially therefrom.

In certain embodiments, the substitutions at amino acid positions 59 and/or 339 are threonine (T) or valine (V). In particular embodiments, the modification are one or more substitutions selected from the group consisting of 59T and 339V. In a further embodiment, the substitutions may comprise the 59T substitution. In a still further embodiment, the substitutions may comprise an additional substitution of 339V.

In some embodiments, the native CGL may be a human CGL. In a particular embodiment, the substitutions can include a combination of E59T and E339V of human CGL (for example, the modified polypeptide having the amino acid sequence of SEQ ID NO: 2, a fragment or homolog thereof). In further embodiments, the modified polypeptide may be a Pongo abelii CGL-TV mutant (SEQ ID NO: 3), Macaca fascicularis CGL-TV mutant (SEQ ID NO: 4), Pan troglodytes CGL-TV mutant (SEQ ID NO: 5), or Pan paniscus CGL-TV mutant (SEQ ID NO: 6).

A modified polypeptide as discussed above may be characterized as having a certain percentage of identity as compared to an unmodified polypeptide (e.g., a native polypeptide) or to any polypeptide sequence disclosed herein. For example, the unmodified polypeptide may comprise at least or up to about 10, 20, 30, 40, 50, 60, 70, 80, 90, 100, 150, 200, 250, 300, 350, 400, 405 residues (or any range derivable therein) of a native primate CGL (i.e., human, Pongo abelii, Macaca fascicularis, Pan troglogytes, or Pan paniscus CGL). The percentage identity may be about, at least 60%, 65%, 70%, 75%, 80%, 85%, 90%, 95%, 96%, 97%, 98%, 99% or 100% (or any range derivable therein) between the unmodified portions of a modified polypeptide (i.e., the sequence of the modified polypeptide excluding any substitutions at amino acids 59 and/or 339) and the corresponding native polypeptide. It is also contemplated that percentage of identity discussed above may relate to a particular modified region of a polypeptide as compared to an unmodified region of a polypeptide. For instance, a polypeptide may contain a modified or mutant substrate recognition site of CGL that can be characterized based on the identity of the amino acid sequence of the modified or mutant substrate recognition site of CGL to that of an unmodified or mutant CGL from the same species or across species. For example, a modified or mutant human polypeptide characterized as having at least 90% identity to an unmodified CGL means that at least 90% of the amino acids in that modified or mutant human polypeptide are identical to the amino acids in the unmodified polypeptide.

In some aspects, the present invention also contemplates polypeptides comprising the modified CGL linked to a heterologous amino acid sequence. For example, the modified CGL may be linked to the heterologous amino acid sequence as a fusion protein. In a particular embodiment, the modified CGL may be linked to amino acid sequences, such as an IgG Fc, albumin, an albumin binding peptide, or an XTEN polypeptide for increasing the in vivo half-life.

To increase serum stability, the modified CGL may be linked to one or more polyether molecules. In a particular embodiment, the polyether may be polyethylene glycol (PEG). The modified polypeptide may be linked to PEG via specific amino acid residues, such as lysine or cysteine. For therapeutic administration, such a polypeptide comprising the modified CGL may be dispersed in a pharmaceutically acceptable carrier.

In some aspects, a nucleic acid encoding such a modified CGL is contemplated. In one aspect, the nucleic acid has been codon optimized for expression in bacteria. In particular embodiments, the bacteria is E. coli. In other aspects, the nucleic acid has been codon optimized for expression in a fungus (e.g., yeast), in insect cells, or in mammalian cells. The present invention further contemplates vectors, such as expression vectors, containing such nucleic acids. In particular embodiments, the nucleic acid encoding the modified CGL is operably linked to a promoter, including but not limited to heterologous promoters. In one embodiment, a modified CGL may be delivered to a target cell by a vector (e.g., a gene therapy vector). Such viruses may have been modified by recombinant DNA technology to enable the expression of the modified CGL-encoding nucleic acid in the target cell. These vectors may be derived from vectors of non-viral (e.g., plasmids) or viral (e.g., adenovirus, adeno-associated virus, retrovirus, lentivirus, herpes virus, or vaccinia virus) origin. Non-viral vectors are preferably complexed with agents to facilitate the entry of the DNA across the cellular membrane. Examples of such non-viral vector complexes include the formulation with polycationic agents which facilitate the condensation of the DNA and lipid-based delivery systems. An example of a lipid-based delivery system would include liposome based delivery of nucleic acids.

In still further aspects, the present invention further contemplates host cells comprising such vectors. The host cells may be bacteria (e.g., E. coli), fungal cells (e.g., yeast), insect cells, or mammalian cells.

In some embodiments, the vectors are introduced into host cells for expressing the modified CGL. The proteins may be expressed in any suitable manner. In one embodiment, the proteins are expressed in a host cell such that the protein is glycosylated. In another embodiment, the proteins are expressed in a host cell such that the protein is aglycosylated.

In some embodiments, the polypeptides or nucleic acids are in a pharmaceutical formulation comprising a pharmaceutically acceptable carrier. The polypeptide may be a native primate CGL polypeptide or a modified CGL polypeptide. The nucleic acid may encode a native primate CGL polypeptide or a modified CGL polypeptide.

In one embodiment, methods are provided for treating a subject having or at risk of developing cystinuria comprising administering to the subject a therapeutically effective amount of a formulation comprising an isolated, modified primate cystathionine-γ-lyase (CGL) enzyme having at least two substitutions relative to a native primate CGL amino acid sequence (see SEQ ID NOs: 1 and 7-10), said at least two substitutions including a threonine at position 59 and a valine at position 339 of the native primate CGL sequence or a nucleic acid comprising a nucleotide sequence encoding the isolated, modified primate cystathionine-γ-lyase (CGL) enzyme. In some aspects, the enzyme further comprises a heterologous peptide segment, such as an XTEN peptide, an IgG Fc, an albumin, or an albumin binding peptide. In some aspects, the enzyme is coupled to polyethylene glycol (PEG). In some aspects, the enzyme is coupled to PEG via one or more lysine or cystine residues.

The subject may be any animal, such as a mouse. For example, the subject may be a mammal, particularly a primate, and more particularly a human patient. In certain aspects, the subject or patient may be maintained on a methionine-restricted diet or a normal diet.

In some aspects, the subject has previously been treated for cystinuria and the enzyme is administered to prevent the recurrence of cystinuria. In some aspects, the method further comprises administering at least a second cystinuria therapy to the subject. In some aspects, the second cystinuria therapy is a surgical therapy or a shock wave therapy.

Certain aspects of the present invention also contemplate methods of treatment by the administration of the native primate CGL peptide, the nucleic acid encoding the native primate CGL peptide in a gene therapy vector, the modified CGL peptide, the nucleic acid encoding the modified CGL in a gene therapy vector, or the formulation of the present invention, and in particular methods of treating tumor cells or subjects with cancer. The subject may be any animal, such as a mouse. For example, the subject may be a mammal, particularly a primate, and more particularly a human patient. In some embodiments, the method may comprise selecting a patient with cancer. In certain aspects, the subject or patient may be maintained on a L-cyst(e)ine-restricted diet or a normal diet.

In some embodiments, the cancer is any cancer that is sensitive to L-cyst(e)ine depletion. In one embodiment, the present invention contemplates a method of treating a tumor cell or a cancer patient comprising administering a formulation comprising such a polypeptide. In some embodiments, the administration occurs under conditions such that at least a portion of the cells of the cancer are killed. In another embodiment, the formulation comprises such a modified CGL with L-cyst(e)ine degrading activity at physiological conditions and further comprising an attached polyethylene glycol chain. In some embodiment, the formulation is a pharmaceutical formulation comprising any of the above discussed CGL variants and pharmaceutically acceptable excipients. Such pharmaceutically acceptable excipients are well known to those of skill in the art. All of the above CGL variants may be contemplated as useful for human therapy.

In a further embodiment, there may also be provided a method of treating a tumor cell comprising administering a formulation comprising a non-bacterial (mammalian, e.g., primate or mouse) modified CGL that has L-cyst(e)ine degrading activity or a nucleic acid encoding thereof.

Because tumor cells are dependent upon their nutrient medium for L-cyst(e)ine, the administration or treatment may be directed to the nutrient source for the cells, and not necessarily the cells themselves. Therefore, in an in vivo application, treating a tumor cell includes contacting the nutrient medium for a population of tumor cells with the engineered (i.e., modified) CGL. In this embodiment, the medium can be blood, lymphatic fluid, spinal fluid and the like bodily fluid where L-cyst(e)ine depletion is desired.

In accordance with certain aspects of the present invention, such a formulation containing the modified CGL can be administered intravenously, intradermally, intraarterially, intraperitoneally, intralesionally, intracranially, intraarticularly, intraprostaticaly, intrapleurally, intrasynovially, intratracheally, intranasally, intravitreally, intravaginally, intrarectally, intratumorally, intramuscularly, subcutaneously, subconjunctival, intravesicularlly, mucosally, intrapericardially, intraumbilically, intraocularly, orally, topically, by inhalation, infusion, continuous infusion, localized perfusion, via a catheter, via a lavage, in lipid compositions (e.g., liposomes), or by other method or any combination of the forgoing as would be known to one of ordinary skill in the art.

In a further embodiment, the method may also comprise administering at least a second anticancer therapy to the subject. The second anticancer therapy may be a surgical therapy, chemotherapy, radiation therapy, cryotherapy, hormone therapy, immunotherapy or cytokine therapy.

In one embodiment, a composition comprising a modified CGL or a nucleic acid encoding a modified CGL is provided for use in the treatment of a tumor in a subject. In another embodiment, the use of a modified CGL or a nucleic acid encoding a modified CGL in the manufacture of a medicament for the treatment of a tumor is provided. Said modified CGL may be any modified CGL of the embodiments.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding,” with reference to a nucleic acid, are used to make the invention readily understandable by the skilled artisan; however, these terms may be used interchangeably with “comprise” or “comprising,” respectively.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Analysis of number of cystine crystals in spot collection urine over the course of the dosing schedule.

FIG. 2. Analysis of number of cystine crystals in spot collection urine pre- and post-dosing.

FIG. 3. Analysis of urinary cysteine/creatinine levels over the course of the dosing schedule.

### DESCRIPTION OF ILLUSTRATIVE EMBODIMENTS

Cysteine is considered a non-essential amino acid as it can be synthesized from homocysteine derived from the essential amino acid L-methionine via the transsulfuration pathway, which comprises the enzymes cystathionine-β-synthase (CBS) and cystathionine-γ-lyase (CGL). Thus, the depletion of cysteine is expected to be relatively non-toxic to normal tissues with an intact transsulfuration pathway.

Cystinuria is a hereditary disorder caused by mutations in the SLC3A1 and SLC7A9 genes encoding the kidney proximal tubule's cystine and dibasic amino acid transporter that leads to abnormal excretion of cystine (the disulfide form of the amino acid cysteine) and the formation of cystine crystals/stones in the urinary tract. Several mouse models of cystinuria are available, including a Slc7a9 knockout mouse, a Slc3a1 knockout mouse, a D140G Slc3a1 mutant mouse, and a E383K Slc3a1 mutant mouse (Feliubadalo et al., 2003; Ercolani et al., 2010; Peters et al., 2003; Livrozet et al., 2014, each of which is incorporated herein by reference in its entirety). Sequence analysis of Slc3a1 genomic DNA from 129S2/SvPasCrl revealed a homozygous mutation in exon 7 in 129S2/SvPasCrl mice. The point A1232G mutation is a missense mutation (c.1232G>A) in a highly conserved sequence. As a consequence, the glutamine in position 383 would be substituted for a lysine (p. E383K). This substitution stands in the extracellular part of rBAT and is responsible for the loss of rBAT expression and cystinuria in129S2/SvPasCrl mice (Livrozet et al., 2014). The glutamine in position 383 is highly conserved among various species.

Patients with cystinuria have a low quality of life, a life-long risk of cystine stone formation, impaired renal function and often require repeated surgical interventions. There are no existing curative therapies for cystinuria and treatments are directed at increasing cystine solubility and lowering urinary cystine concentrations. Hyperdiuresis is a common treatment, however it requires daily consumption of >4 liters of water and urine volumes >3 liters which is difficult to achieve and maintain. Other drug treatments, such as small thiol molecules, function by reacting with cystine to form mixed disulfides that are more soluble than cystine but have significant toxicities, such as such as leukopenia, rash, fever, proteinuria and nephritic syndrome, which limit their use.

The present invention provides methods of using engineered, therapeutic enzymes that degrade L-cyst(e)ine to treat diseases, such as cystinuria. This method removes cystine from circulation which has been shown clinically to reduce the incidence of kidney and urinary cystine stone formation in cystinuria patients. The method described here can reduce circulating cystine below detection levels without the side-effects associated with current cystinuria drugs.

### I. DEFINITIONS

As used herein the terms “protein” and “polypeptide” refer to compounds comprising amino acids joined via peptide bonds and are used interchangeably.

As used herein, the term “fusion protein” refers to a chimeric protein containing proteins or protein fragments operably linked in a non-native way.

As used herein, the term “half-life” (½-life) refers to the time that would be required for the concentration of a polypeptide thereof to fall by half in vitro or in vivo, for example, after injection in a mammal.

The terms “in operable combination,” “in operable order,” and “operably linked” refer to a linkage wherein the components so described are in a relationship permitting them to function in their intended manner, for example, a linkage of nucleic acid sequences in such a manner that a nucleic acid molecule capable of directing the transcription of a given gene and/or the synthesis of desired protein molecule, or a linkage of amino acid sequences in such a manner so that a fusion protein is produced.

The term “linker” is meant to refer to a compound or moiety that acts as a molecular bridge to operably link two different molecules, wherein one portion of the linker is operably linked to a first molecule, and wherein another portion of the linker is operably linked to a second molecule.

The term “PEGylated” refers to conjugation with polyethylene glycol (PEG), which has been widely used as a drug carrier, given its high degree of biocompatibility and ease of modification. PEG can be coupled (e.g., covalently linked) to active agents through the hydroxy groups at the end of the PEG chain via chemical methods; however, PEG itself is limited to at most two active agents per molecule. In a different approach, copolymers of PEG and amino acids have been explored as novel biomaterial that would retain the biocompatibility of PEG, but that would have the added advantage of numerous attachment points per molecule (thus providing greater drug loading), and that can be synthetically designed to suit a variety of applications.

The term “gene” refers to a DNA sequence that comprises control and coding sequences necessary for the production of a polypeptide or precursor thereof. The polypeptide can be encoded by a full-length coding sequence or by any portion of the coding sequence so as the desired enzymatic activity is retained.

The term “native” refers to the typical form of a gene, a gene product, or a characteristic of that gene or gene product when isolated from a naturally occurring source. A native form is that which is most frequently observed in a natural population and is thus arbitrarily designated the normal or wild-type form. In contrast, the term “modified,” “variant,” or “mutant” refers to a gene or gene product that displays modification in sequence and functional properties (i.e., altered characteristics) when compared to the native gene or gene product.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

The term “therapeutically effective amount” as used herein refers to an amount of a therapeutic composition (such as a therapeutic polynucleotide and/or therapeutic polypeptide) that is employed in methods to achieve a therapeutic effect. The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cystinuria may involve, for example, a reduction in the size of a cystine stone, elimination of a cystine stone, or prevention of the formation of a cystine stone.

The term “K_{M}” as used herein refers to the Michaelis-Menten constant for an enzyme and is defined as the concentration of the specific substrate at which a given enzyme yields one-half its maximum velocity in an enzyme catalyzed reaction. The term “k_{cat}” as used herein refers to the turnover number or the number of substrate molecules each enzyme site converts to product per unit time, and in which the enzyme is working at maximum efficiency. The term “k_{cat}/K_{M}” as used herein is the specificity constant, which is a measure of how efficiently an enzyme converts a substrate into product.

The term “cystathionine-γ-lyase” (CGL or cystathionase) refers to any enzyme that catalyzes the hydrolysis of cystathionine to cysteine. For example, it includes primate forms of cystathionine-γ-lyase, or particularly, human forms of cystathionine-γ-lyase.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of a cyst(e)inease.

“Subject” and “patient” refer to either a human or non-human, such as primates, mammals, and vertebrates. In particular embodiments, the subject is a human.

### II. CYSTATHIONINE-γ-LYASE

A lyase is an enzyme that catalyzes the breaking of various chemical bonds, often forming a new double bond or a new ring structure. For example, an enzyme that catalyzed this reaction would be a lyase: ATP→cAMP+PPᵢ. Lyases differ from other enzymes in that they only require one substrate for the reaction in one direction, but two substrates for the reverse reaction.

A number of pyrioxal-5′-phosphate (PLP)-dependent enzymes are involved in the metabolism of cysteine, homocysteine, and methionine, and these enzymes form an evolutionary related family, designated as Cys/Met metabolism PLP-dependent enzymes. These enzymes are proteins of about 400 amino acids and the PLP group is attached to a lysine residue located in the central location of the polypeptide. Members of this family include cystathionine-γ-lyase (CGL), cystathionine-γ-synthase (CGS), cystathionine-β-lyase (CBL), methionine-γ-lyase (MGL), O-acetylhomoserine (OAH)/O-acetyl-serine (OAS) sulfhydrylase (OSHS). Common to all of them is the formation of a Michaelis complex leading to an external substrate aldimine. The further course of the reaction is determined by the substrate specificity of the particular enzyme.

For example, the inventors introduced specific mutations into a PLP-dependent lyase family member, cystathionine-γ-lyase, to change its substrate specificity. In this manner, variants were produced with the de novo ability to degrade both L-cystine and L-cysteine. In other embodiments, a modification of other PLP-dependent enzymes for producing novel L-cyst(e)ine degrading activity may also be contemplated.

Cystathionine-γ-lyase (CGL or cystathionase) is an enzyme which breaks down cystathionine into cysteine and α-ketobutyrate. Pyridoxal phosphate is a prosthetic group of this enzyme. Protein engineering was used to convert cystathionase, which has only weak activity for the degradation of L-cysteine and L-cystine, into an enzyme that can degrade these amino acids at a high rate (U.S. patent application Ser. No. 14/472,779, which is incorporated herein by reference in its entirety).

### II. CYST(E)INEASE ENGINEERING

Due to the undesired effects of immunogenicity seen clinically with the use of non-human protein therapeutics, the inventors sought to engineer therapeutically relevant cystine/cysteine degrading activity into a human enzyme (i.e., engineer an enzyme with high k_{cat} and low K_{M} values for cystine/cysteine and also displaying a favorable specificity). Humans have an enzyme called cystathionine-γ-lyase (hCGL) whose function is to catalyze the last step in the mammalian transsulfuration pathway (Rao et al., 1990), namely the conversion of L-cystathionine to L-cysteine, alpha-ketobutyrate, and ammonia. Human CGL can also weakly degrade L-cysteine and its disulfide form, L-cystine, making it an ideal candidate for engineering. Using structurally- and phylogenetically-guided mutagenesis, hCGL variants were engineered to efficiently hydrolyze both L-cysteine and L-cystine.

Some embodiments concern modified proteins and polypeptides. Particular embodiments concern a modified protein or polypeptide that exhibits at least one functional activity that is comparable to the unmodified version, preferably, the L-cyst(e)ine degrading activity. In further aspects, the protein or polypeptide may be further modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide, such as the L-cyst(e)ine degrading activity. In certain embodiments, the unmodified protein or polypeptide is a native CGL, preferably a human CGL. It is specifically contemplated that embodiments concerning a “modified protein” may be implemented with respect to a “modified polypeptide,” and vice versa.

Determination of activity may be achieved using assays familiar to those of skill in the art, particularly with respect to the protein's activity, and may include for comparison purposes, for example, the use of native and/or recombinant versions of either the modified or unmodified protein or polypeptide. For example, Human CGL slowly degrades L-cysteine to pyruvate, ammonia and H₂S, and converts L-cystine to pyruvate, ammonia and thiocysteine (k_{cat}/K_{M}˜0.2 s⁻¹ mM⁻¹ and 0.5 s⁻¹ mM⁻¹, respectively). Thiocysteine is further nonenzymatically degraded to L-cysteine and H₂S. Thus, the L-cyst(e)ine degrading activity may be determined by any assay to detect the production of any substrates resulting from the degradation of L-cystine and/or L-cysteine, such as the detection of pyruvate using 3-methyl-2-benzothiazolinone hydrazone (MBTH) (Takakura et al., 2004).

In certain embodiments, a modified polypeptide, such as a modified CGL, may be identified based on its increase in L-cyst(e)ine degrading activity. For example, substrate recognition sites of the unmodified polypeptide may be identified. This identification may be based on structural analysis or homology analysis. A population of mutants involving modifications of such substrate recognitions sites may be generated. In a further embodiment, mutants with increased L-cyst(e)ine degrading activity may be selected from the mutant population. Selection of desired mutants may include methods for the detection of byproducts or products from L-cyst(e)ine degradation.

Modified proteins may possess deletions and/or substitutions of amino acids; thus, a protein with a deletion, a protein with a substitution, and a protein with a deletion and a substitution are modified proteins. In some embodiments, these modified proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability.

Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 900/%, or even between about 91% and about 99% of amino acids that are identical or functionally equivalent to the amino acids of a control polypeptide are included, provided the biological activity of the protein is maintained. A modified protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

One particular variant identified as having the highest catalytic activity for degrading both L-cystine and L-cysteine was found to have the following mutations: E59T, a synonymous codon change of R 19R, and E339V. This variant was called hCGL-TV and was characterized for its ability to degrade L-cyst(e)ine in a 100 mM PBS buffer at pH 7.3 and 37° C. using a 1 mL scale MBTH assay similar to that described above. Under these conditions, the hCGL-TV variant was found to degrade L-cystine with a k_{cat} of 1.0±0.05 s⁻¹, a K_{M} of 0.16±0.02 mM, and a k_{cat}/K_{M} of 6.3±1.0 s⁻¹ mM⁻¹. The hCGL-TV variant was further found to have a k_{cat} of 0.8±0.03 s⁻¹, a K_{M} of 0.25±0.04 mM, and a k_{cat}/K_{M} of 3.2±0.6 s⁻¹ mM⁻¹ for degradation of L-cysteine. The hCGL-TV variant was found to have very high stability in human serum with an apparent T_{0.5} of 228±6 h. In addition, the hCGL-TV was found to have an apparent IC₅₀ value of ˜60 nM for both the DU145 and PC3 prostate tumor cell lines.

### IV. ENZYMATIC L-CYST(E)INE DEGRADATION FOR THERAPY

In certain aspects, the polypeptides may be used for the treatment of diseases, such as cystinuria, with novel enzymes that deplete L-cystine and/or L-cysteine. The invention specifically discloses treatment methods using modified CGL with L-cyst(e)ine degrading activity. Certain embodiments of the present invention provide novel enzymes with L-cyst(e)ine degrading activity for increased therapeutic efficacy.

Certain aspects of the present invention provide a modified CGL with L-cyst(e)ine degrading activity for treating diseases. In one example, the modified polypeptide may have human polypeptide sequences and thus may prevent allergic reactions in human patients, allow repeated dosing, and increase the therapeutic efficacy.

As an example, PEG-hCGL-TV can drastically reduce serum cystine levels (>95%) over 96 h and cysteine levels (80%) over 48 h. To determine this, male FVB mice were injected i.p. with 50 mg/kg of PEG-hCGL-TV and sacrificed at days 0, 1, 2, 4, and 6 (n=5 per group) for blood and serum collection. Serum samples were mixed with an internal standard mixture of 10 picomole deuterated cystine and cysteine and ultrafiltered using NANOSEP® OMEGA™ centrifugal devices, 3 kDa cutoff (Pall Life Biosciences) (Tiziani et al., 2008; Tiziani et al., 2013). The filtered polar fractions were chromatographed using a reverse-phase BEH C18, 1.7 μm, 2.1×150 mm column (THERMO SCIENTIFIC™ ACCELA® 1250 UPLC, Waters Corporation, USA) and introduced into an EXACTIVE™ Plus ORBITRAP™ mass spectrometer coupled with electrospray ionization (Thermo Fisher Scientific, San Jose, Calif.). Data was acquired in centroid MS mode from 50 to 700 m/z mass range with the XCALIBUR™ software provided with instrument. The relative concentrations of cystine and cysteine are reported as mean values±SEM.

In addition, PEG-hCGL-TV demonstrated an absorption T_{1/2} of approximately 23 h, and an elimination T_{1/2} of 40±7 h. To determine this, a dot blot densitometry technique was used where samples were probed with an anti-hCGL antibody (rabbit anti-CTH Sigma # C8248) followed by addition of anti-rabbit IgG-FITC (Santa Cruz Biotechnology # sc-2012) for visualization by excitation at 488 nm on a TYPHOON™ scanner (GE Healthcare). Using ImageJ software (Schneider et al., 2012), densitometry bands of the samples were compared to titrations of known amounts of PEG-hCGL-TV within the same blot to construct a standard curve and calculate relative serum PEG-hCGL-TV levels. The data were fit to an extravascular model of administration (Foye et al., 2007; Stone et al., 2012).

Depletion can be conducted in vivo in the circulation of a mammal, in vitro in cases where L-cystine and/or L-cysteine depletion in tissue culture or other biological mediums is desired, and in ex vivo procedures where biological fluids, cells, or tissues are manipulated outside the body and subsequently returned to the body of the patient mammal. Depletion of L-cystine and/or L-cysteine from circulation, culture media, biological fluids, or cells is conducted to reduce the amount of L-cystine and/or L-cysteine accessible to the material being treated, and therefore comprises contacting the material to be depleted with a L-cystine- and/or L-cysteine-degrading amount of the engineered primate cyst(e)inease under L-cystine- and/or L-cysteine-degrading conditions as to degrade the ambient L-cystine and/or L-cysteine in the material being contacted.

L-cystine- and/or L-cysteine-degrading efficiency can vary widely depending upon the application, and typically depends upon the amount of L-cystine and/or L-cysteine present in the material, the desired rate of depletion, and the tolerance of the material for exposure to cyst(e)inease. L-cystine and/or L-cysteine levels in a material, and therefore rates of L-cystine and/or L-cysteine depletion from the material, can readily be monitored by a variety of chemical and biochemical methods well known in the art. Exemplary L-cystine- and/or L-cysteine-degrading amounts are described further herein, and can range from 0.001 to 100 units (U) of engineered cyst(e)inease, preferably about 0.01 to 10 U, and more preferably about 0.1 to 5 U engineered cyst(e)inease per milliliter (mL) of material to be treated.

L-cystine- and/or L-cysteine-degrading conditions are buffer and temperature conditions compatible with the biological activity of a CGL enzyme, and include moderate temperature, salt, and pH conditions compatible with the enzyme, for example, physiological conditions. Exemplary conditions include about 4-40° C., ionic strength equivalent to about 0.05 to 0.2 M NaCl, and a pH of about 5 to 9, while physiological conditions are included.

In one embodiment, the contacting in vivo is accomplished by administering, by intravenous or intraperitoneal injection, a therapeutically effective amount of a physiologically tolerable composition comprising an engineered cyst(e)inease of this invention to a patient, thereby depleting the circulating L-cystine and/or L-cysteine present in the patient.

A therapeutically effective amount of an engineered cyst(e)inease is a predetermined amount calculated to achieve the desired effect, i.e., to deplete L-cystine and/or L-cysteine in a patient's circulation. Thus, the dosage ranges for the administration of engineered cyst(e)inease of the invention are those large enough to produce the desired effect. The dosage should not be so large as to cause adverse side effects, such as hyperviscosity syndromes, pulmonary edema, congestive heart failure, and the like. Generally, the dosage will vary with age of, condition of, sex of, and extent of the disease in the patient and can be determined by one of skill in the art. The dosage can be adjusted by the individual physician in the event of any complication.

For example, a therapeutically effective amount of an engineered cyst(e)inease may be an amount such that when administered in a physiologically tolerable composition is sufficient to achieve a intravascular (plasma) or local concentration of from about 0.001 to about 100 units (U) per mL, preferably above about 0.1 U, and more preferably above 1 U engineered cyst(e)inease per mL. Typical dosages can be administered based on body weight, and are in the range of about 5-1000 U/kilogram (kg)/day, preferably about 5-100 U/kg/day, more preferably about 10-50 U/kg/day, and more preferably about 20-40 U/kg/day.

The engineered cyst(e)inease can be administered parenterally by injection or by gradual infusion over time. The engineered cyst(e)inease can be administered intravenously, intraperitoneally, orally, intramuscularly, subcutaneously, intracavity, transdermally, dermally, can be delivered by peristaltic means, can be injected directly into the urinary tract, or can be administered by a pump connected to a catheter that may contain a potential biosensor or L-cyst(e)ine.

The therapeutic compositions containing engineered cyst(e)inease are conventionally administered intravenously, as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for initial administration and booster shots are also contemplated and are typified by an initial administration followed by repeated doses at one or more hour intervals by a subsequent injection or other administration. Exemplary multiple administrations are described herein and are particularly preferred to maintain continuously high serum and tissue levels of engineered cyst(e)inease and conversely low serum and tissue levels of L-cyst(e)ine. Alternatively, continuous intravenous infusion sufficient to maintain concentrations in the blood in the ranges specified for in vivo therapies are contemplated.

### V. CONJUGATES

Compositions and methods of the present invention involve engineered cyst(e)ineases, such as by forming conjugates with heterologous peptide segments or polymers, such as polyethylene glycol. In further aspects, the engineered cyst(e)ineases may be linked to PEG to increase the hydrodynamic radius of the enzyme and hence increase the serum persistence. In certain aspects, the disclosed polypeptide may be conjugated to any targeting agent, such as a ligand having the ability to specifically and stably bind to an external receptor or binding site on a target cell (e.g., U.S. Patent Publ. 2009/0304666).

A. Fusion Proteins

Certain embodiments of the present invention concern fusion proteins. These molecules may have the modified cystathionase linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

In a particular embodiment, the cyst(e)inease may be linked to a peptide that increases the in vivo half-life, such as an XTEN polypeptide (Schellenberger et al., 2009), IgG Fc domain, albumin, or albumin binding peptide.

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

B. Linkers

In certain embodiments, the engineered cyst(e)inease may be chemically conjugated using bifunctional cross-linking reagents or fused at the protein level with peptide linkers.

Bifunctional cross-linking reagents have been extensively used for a variety of purposes, including preparation of affinity matrices, modification and stabilization of diverse structures, identification of ligand and receptor binding sites, and structural studies. Suitable peptide linkers may also be used to link the engineered cyst(e)inease, such as Gly-Ser linkers.

Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidine-, indole-, carboxyl-specific groups. Of these, reagents directed to free amino groups have become especially popular because of their commercial availability, ease of synthesis, and the mild reaction conditions under which they can be applied.

A majority of heterobifunctional cross-linking reagents contain a primary amine-reactive group and a thiol-reactive group. In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling, in one example, of aldehydes to free thiols. The cross-linking reagent can be modified to cross-link various functional groups.

Additionally, any other linking/coupling agents and/or mechanisms known to those of skill in the art may be used to combine primate engineered cyst(e)inease, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo. These linkers are thus one group of linking agents.

In addition to hindered cross-linkers, non-hindered linkers also can be employed in accordance herewith. Other useful cross-linkers, not considered to contain or generate a protected disulfide, include SATA, SPDP, and 2-iminothiolane (Wawrzynczak and Thorpe, 1987). The use of such cross-linkers is well understood in the art. Another embodiment involves the use of flexible linkers.

Once chemically conjugated, the peptide generally will be purified to separate the conjugate from unconjugated agents and from other contaminants. A large number of purification techniques are available for use in providing conjugates of a sufficient degree of purity to render them clinically useful.

Purification methods based upon size separation, such as gel filtration, gel permeation, or high performance liquid chromatography, will generally be of most use. Other chromatographic techniques, such as Blue-Sepharose separation, may also be used. Conventional methods to purify the fusion proteins from inclusion bodies may be useful, such as using weak detergents, such as sodium N-lauroyl-sarcosine (SLS).

C. PEGylation

In certain aspects of the invention, methods and compositions related to PEGylation of engineered cyst(e)inease are disclosed. For example, the engineered cyst(e)inease may be PEGylated in accordance with the methods disclosed herein.

PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of the PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increase in need for heterobifunctional PEGs for conjugation. These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and NHS esters.

The most common modification agents, or linkers, are based on methoxy PEG (mPEG) molecules. Their activity depends on adding a protein-modifying group to the alcohol end. In some instances polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five key parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70 percent active, the amount of PEG used should ensure that only active PEG molecules are counted in the protein-to-PEG reaction stoichiometry.

### VI. PROTEINS AND PEPTIDES

In certain embodiments, the present invention concerns novel compositions comprising at least one protein or peptide, such as an engineered cyst(e)inease. These peptides may be comprised in a fusion protein or conjugated to an agent as described supra.

As used herein, a protein or peptide generally refers, but is not limited to, a protein of greater than about 200 amino acids, up to a full length sequence translated from a gene; a polypeptide of greater than about 100 amino acids; and/or a peptide of from about 3 to about 100 amino acids. For convenience, the terms “protein,” “polypeptide,” and “peptide” are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The nucleotide and protein, polypeptide, and peptide sequences corresponding to various genes have been previously disclosed, and may be found at computerized databases known to those of ordinary skill in the art. One such database is the National Center for Biotechnology Information's Genbank and GenPept databases (available on the world wide web at ncbi.nlm.nih.gov/). The coding regions for known genes may be amplified and/or expressed using the techniques disclosed herein or as would be known to those of ordinary skill in the art. Alternatively, various commercial preparations of proteins, polypeptides, and peptides are known to those of skill in the art.

### VII. NUCLEIC ACIDS AND VECTORS

In certain aspects of the invention, nucleic acid sequences encoding an engineered cyst(e)inease or a fusion protein containing a modified cyst(e)inease may be disclosed. Depending on which expression system is used, nucleic acid sequences can be selected based on conventional methods. For example, if the engineered cyst(e)inease is derived from primate CGL and contains multiple codons that are rarely utilized in E. coli, then that may interfere with expression. Therefore, the respective genes or variants thereof may be codon optimized for E. coli expression. Various vectors may be also used to express the protein of interest, such as engineered cyst(e)inease. Exemplary vectors include, but are not limited, plasmid vectors, viral vectors, transposon, or liposome-based vectors.

### VIII. HOST CELLS

Host cells may be any that may be transformed to allow the expression and secretion of engineered cyst(e)inease and conjugates thereof. The host cells may be bacteria, mammalian cells, yeast, or filamentous fungi. Various bacteria include Escherichia and Bacillus. Yeasts belonging to the genera Saccharomyces, Kiuyveromyvces, Hansenula, or Pichia would find use as an appropriate host cell. Various species of filamentous fungi may be used as expression hosts, including the following genera: Aspergillus, Trichoderma, Neurospora, Penicillium, Cephalosporium, Achlya, Podospora, Endothia, Mucor, Cochliobolus, and Pyricularia.

Examples of usable host organisms include bacteria, e.g., Escherichia coli MC1061, derivatives of Bacillus subtilis BRB1 (Sibakov et al., 1984), Staphylococcus aureus SA1123 (Lordanescu, 1975) or Streptococcus lividans (Hopwood et al., 1985); yeasts, e.g., Saccharomyces cerevisiae AH 22 (Mellor et al., 1983) or Schizosaccharomyces pombe; and filamentous fungi, e.g., Aspergillus nidulans, Aspergilhus awamori (Ward, 1989), or Trichoderma reesei (Penttila et al., 1987; Harkki et al., 1989).

Examples of mammalian host cells include Chinese hamster ovary cells (CHO-K1; ATCC CCL61), rat pituitary cells (GH1; ATCC CCL82), HeLa S3 cells (ATCC CCL2.2), rat hepatoma cells (H-4-II-E; ATCCCRL 1548), SV40-transformed monkey kidney cells (COS-1; ATCC CRL 1650), and murine embryonic cells (NIH-3T3; ATCC CRL 1658). The foregoing being illustrative but not limitative of the many possible host organisms known in the art. In principle, all hosts capable of secretion can be used whether prokaryotic or eukaryotic.

Mammalian host cells expressing the engineered cyst(e)inease and/or their fusion proteins are cultured under conditions typically employed to culture the parental cell line. Generally, cells are cultured in a standard medium containing physiological salts and nutrients, such as standard RPML MEM, IMEM, or DMEM, typically supplemented with 5%-10% serum, such as fetal bovine serum. Culture conditions are also standard, e.g., cultures are incubated at 37° C. in stationary or roller cultures until desired levels of the proteins are achieved.

### IX. PROTEIN PURIFICATION

Protein purification techniques are well known to those of skill in the art.

These techniques involve, at one level, the homogenization and crude fractionation of the cells, tissue, or organ to polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure peptide are ion-exchange chromatography, gel exclusion chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC).

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various techniques suitable for use in protein purification are well known to those of skill in the art. These include, for example, precipitation with ammonium sulphate, PEG, antibodies and the like, or by heat denaturation, followed by centrifugation; chromatography steps, such as ion exchange, gel filtration, reverse phase, hydroxyapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

There is no general requirement that the protein or peptide will always be provided in its most purified state. Indeed, it is contemplated that less substantially purified products may have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

In certain embodiments a protein or peptide may be isolated or purified, for example, an engineered cyst(e)inease, a fusion protein containing the engineered AAD, or an engineered cyst(e)inease post PEGylation. For example, a His tag or an affinity epitope may be comprised in such an engineered cyst(e)inease to facilitate purification. Affinity chromatography is a chromatographic procedure that relies on the specific affinity between a substance to be isolated and a molecule to which it can specifically bind. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding. It should be possible to elute the substance without destroying the sample or the ligand.

Size exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase.

The underlying principle of SEC is that particles of different sizes will elute (filter) through a stationary phase at different rates. This results in the separation of a solution of particles based on size. Provided that all the particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

High-performance liquid chromatography (or high-pressure liquid chromatography, HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used.

As an example, each construct may contain an N-terminal NcoI restriction site, an in-frame N-terminal His₆ tag and a C-terminal EcoRI site for simplifying cloning. After cloning into a pET28a vector (Novagen), E. coli (BL21) containing an appropriate hCGL expression vector may be grown until reaching an OD₆₀₀ of ˜0.5-0.6. At this point the cultures may be switched to a shaker at 25° C. and induced with 0.5 mM IPTG and allowed to express protein for an additional 12 h. Cell pellets may then be collected by centrifugation and re-suspended in an IMAC buffer (10 mM NaPO₄/10 mM imidazole/300 mM NaCl, pH 8). After lysis by a French pressure cell, lysates may be centrifuged at 20,000×g for 20 min at 4° C., and the resulting supernatant applied to a nickel IMAC column, washed with 10-20 column volumes of IMAC buffer, and then eluted with an IMAC elution buffer (50 mM NaPO₄/250 mM imidazole/300 mM NaCl, pH 8). Fractions containing enzyme may then be incubated with 10 mM pyridoxal phosphate (PLP) for an hour at 25° C. Using a 10,000 MWCO centrifugal filter device (AMICON™), proteins may then be buffer exchanged several times into a 100 mM PBS, 10% glycerol, pH 7.3 solution. Aliquots of hCGL enzyme or hCGL-variant enzyme can be flash frozen in liquid nitrogen and stored at −80° C. hCGL or hCGL-variant enzyme purified in this manner may be >95% homogeneous as assessed by SDS-PAGE and coomassie staining.

### X. PHARMACEUTICAL COMPOSITIONS

It is contemplated that the novel cyst(e)inease can be administered systemically or locally. They can be administered intravenously, intrathecally, and/or intraperitoneally.

It is not intended that the present invention be limited by the particular nature of the therapeutic preparation. For example, such compositions can be provided in formulations together with physiologically tolerable liquid, gel, or solid carriers, diluents, and excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects.

Such compositions are typically prepared as liquid solutions or suspensions, as injectables. Suitable diluents and excipients are, for example, water, saline, dextrose, glycerol, or the like, and combinations thereof. In addition, if desired, the compositions may contain minor amounts of auxiliary substances, such as wetting or emulsifying agents, stabilizing agents, or pH buffering agents.

Where clinical applications are contemplated, it may be necessary to prepare pharmaceutical compositions comprising proteins, antibodies, and drugs in a form appropriate for the intended application. Generally, pharmaceutical compositions may comprise an effective amount of one or more CGL variant or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition that contains at least one CGL variant isolated by the method disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier is incompatible with the active ingredient, its use in the pharmaceutical compositions is contemplated.

Certain embodiments of the present invention may comprise different types of carriers depending on whether it is to be administered in solid, liquid, or aerosol form, and whether it needs to be sterile for the route of administration, such as injection. The compositions can be administered intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference).

The modified polypeptides may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

Further in accordance with certain aspects of the present invention, the composition suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a composition contained therein, its use in administrable composition for use in practicing the methods is appropriate. Examples of carriers or diluents include fats, oils, water, saline solutions, lipids, liposomes, resins, binders, fillers, and the like, or combinations thereof. The composition may also comprise various antioxidants to retard oxidation of one or more component. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

In accordance with certain aspects of the present invention, the composition is combined with the carrier in any convenient and practical manner, i.e., by solution, suspension, emulsification, admixture, encapsulation, absorption, and the like. Such procedures are routine for those skilled in the art.

In a specific embodiment of the present invention, the composition is combined or mixed thoroughly with a semi-solid or solid carrier. The mixing can be carried out in any convenient manner, such as grinding. Stabilizing agents can be also added in the mixing process in order to protect the composition from loss of therapeutic activity, i.e., denaturation in the stomach. Examples of stabilizers for use in a composition include buffers, amino acids, such as glycine and lysine, carbohydrates, such as dextrose, mannose, galactose, fructose, lactose, sucrose, maltose, sorbitol, mannitol, etc.

In further embodiments, the present invention may concern the use of a pharmaceutical lipid vehicle composition that includes CGL variants, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods.

One of ordinary skill in the art would be familiar with the range of techniques that can be employed for dispersing a composition in a lipid vehicle. For example, the engineered cyst(e)inease or a fusion protein thereof may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

As an example, the hCGL-TV enzyme may be purified by washing the bound IMAC column extensively (90-100 column volumes) with an IMAC buffer containing 0.1% TRITON™ 114 in the sample. The sample may be washed again with 10-20 column volumes of IMAC buffer, and then eluted with an IMAC elution buffer (50 mM NaPO₄/250 mM imidazole/300 mM NaCl, pH 8). The wash with TRITON™ 114 may be employed for endotoxin removal. The purified protein may be subjected to buffer exchange into a 100 mM NaPO₄ buffer at pH 8.3 using a 10,000 MWCO filtration device (AMICON®). Subsequently, PLP may be added at a concentration of 10 mM and the protein incubated for 1 h at 25° C. Methoxy PEG Succinimidyl Carboxymethyl Ester 5000 MW (JenKem Technology) may then be added to hCGL-TV at an 80:1 molar ratio and allowed to react for 1 h at 25° C. under constant stirring. The resulting mixture may be extensively buffer exchanged (PBS with 10% glycerol) using a 100,000 MWCO filtration device (AMICON®), and sterilized with a 0.2 micron syringe filter (VWR). All PEGylated enzymes may be analyzed for lipopolysaccharide (LPS) content using a Limulus Amebocyte Lysate (LAL) kit (Cape Cod Incorporated).

### XI. COMBINATION TREATMENTS

In certain embodiments, the compositions and methods of the present embodiments involve administration of a cyst(e)inease in combination with a second or additional therapy. Such therapy can be applied in the treatment of any disease that is associated with cyst(e)ine dependency. For example, the disease may be cystinuria.

The methods and compositions, including combination therapies, enhance the therapeutic or protective effect, and/or increase the therapeutic effect of another therapy. Therapeutic and prophylactic methods and compositions can be provided in a combined amount effective to achieve the desired effect, such as the elimination of cysteine stones in the urinary tract. This process may involve administering both a cyst(e)inease and a second therapy. A tissue, organ, or cell can be exposed to one or more compositions or pharmacological formulation(s) comprising one or more of the agents (i.e., a cyst(e)inease or a second agent), or by contacting the tissue, organ, and/or cell with two or more distinct compositions or formulations, wherein one composition provides 1) a cyst(e)inease, 2) a second agent, or 3) both a cyst(e)inease and a second agent. Also, it is contemplated that such a combination therapy can be used in conjunction with shock wave therapy or surgical therapy.

The terms “contacted” and “exposed,” when applied to a cell, are used herein to describe the process by which a therapeutic construct are delivered to a target organ or are placed in direct juxtaposition with the target cell. To achieve stone dissolution, for example, both agents are delivered to a cell in a combined amount effective to dissolve the stone or prevent it from forming or reforming.

A cyst(e)inease may be administered before, during, after, or in various combinations relative to a second cystinuria treatment. The administrations may be in intervals ranging from concurrently to minutes to days to weeks. In embodiments where the cyst(e)inease is provided to a patient separately from a second cystinuria agent, one would generally ensure that a significant period of time did not expire between the time of each delivery, such that the two treatments would still be able to exert an advantageously combined effect on the patient. In such instances, it is contemplated that one may provide a patient with the cyst(e)inease and the second cystinuria therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that the cyst(e)inease may be given on any day of day 1 to day 90 (this such range includes intervening days) or any combination thereof, and another treatment is given on any day of day 1 to day 90 (this such range includes intervening days) or any combination thereof. Within a single day (24-hour period), the patient may be given one or multiple administrations of the treatment(s). Moreover, after a course of treatment, it is contemplated that there is a period of time at which no treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a cyst(e)inease is “A” and a second cystinuria therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Surgery

One of the most common methods for the removal of cystine stones is percutaneous nephrolithotomy, in which a keyhole incision is made in the back and a nephroscope is used to break up and remove the stones. Although this procedure is less invasive than open surgery, regular or spinal anesthesia is normally required along with a hospital stay of 2 to 3 days and a recovery time of a few weeks.

B. Shock wave therapy

Cystinuric patients often have recurrent episodes of stone formation and surgeries in their lifetime. Shock wave lithotripsy, the use of high-energy shock waves for stone fragmentation, can be used for treatment of cystine stones that are smaller than 1.5 cm. Cystine stones are the most sturdy of all urinary stones and lithotripsy is generally ineffective in breaking them up. However, smaller cystine stones may be fragmented with lithotripsy because more frequent shocks at higher energy can be used.

C. Drug Therapy

Drug therapy involves the use of thiol-containing drugs, such as D-penicillamine, α-mercaptopropionylglycine (Thiola), and captopril, to break the cystine disulfide bond and form more soluble mixed disulfides. However, these drugs frequently give the patient various unpleasant side effects such as gastrointestinal intolerance, rash and pain in the joints (Sakhaee and Sutton, 1996).

D. Other Methods

Additional courses of treatment usually involve management of urinary cystine levels to reduce the risk of stone formation. These management methods include substantially increasing the intake of water (thereby increasing the urine volume and the amount of cystine that can be solubilized), dietary restrictions of methionine, which is a metabolic precursor of cystine, and sodium, and oral administration of potassium citrate to increase the pH of the urine, thereby increasing the solubility of cystine.

An additional method for the treatment of cystine stones, which is a non-surgical and minimally invasive route, involves the delivery of chemical solutions to the kidneys via a nephrostomy catheter for the chemical dissolution of the stones, also known as chemodissolution. A variety of chemolytic agents have been used in this technique including sodium bicarbonate and the organic buffer tris-hydroxymethylene-aminomethane (tromethamine-E) at pH 10, both which act to provide a strongly alkaline environment to dissolve the cystine stones. Acetylcysteine is also frequently used in chemodissolution and dissolves the stones in a manner similar to D-penicillamine and Thiola by breaking the cystine disulfide and forming more soluble disulfides. However, this dissolution method has a limited role in the treatment of cystine stones because these chemolytic agents perform extremely slowly and can typically take weeks to months to dissolve stones (Ng and Streem, 2001).

### XII. KITS

Certain aspects of the present invention may provide kits, such as therapeutic kits. For example, a kit may comprise one or more pharmaceutical composition as described herein and optionally instructions for their use. Kits may also comprise one or more devices for accomplishing administration of such compositions. For example, a subject kit may comprise a pharmaceutical composition and catheter for accomplishing direct intravenous injection of the composition into the urinary tract. In other embodiments, a subject kit may comprise pre-filled ampoules of an engineered cyst(e)inease, optionally formulated as a pharmaceutical, or lyophilized, for use with a delivery device.

Kits may comprise a container with a label. Suitable containers include, for example, bottles, vials, and test tubes. The containers may be formed from a variety of materials, such as glass or plastic. The container may hold a composition that includes an engineered cyst(e)inease that is effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Treatment of a Mouse Model of Cystinuria with a Human Cyst(e)Inease Enzyme

The ability of hCGL-TV to prevent or reverse stone formation in the urinary tract was evaluated in Slc3a1 knockout mice, which have previously been reported to develop cystine stones in the bladder and, to a lesser extent, in the kidney (Ercolani et al., 2010). The Slc3a1 mouse model used for this research project was created from ES cell clone 16054A-G2, generated by Regeneron Pharmaceuticals, Inc. and cloned into mice by the KOMP Repository (www.komp.org) at the University of California Davis (U42RR024244). The line was further bred and provided to the KOMP Repository (www.komp.org) as part of the KOMP2 Project by the Charles River Laboratory as part of the DTCC consortium.

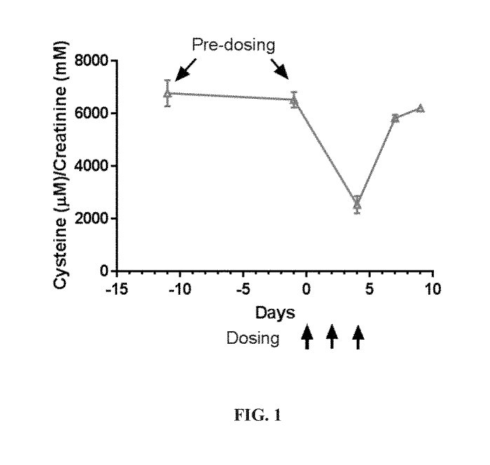

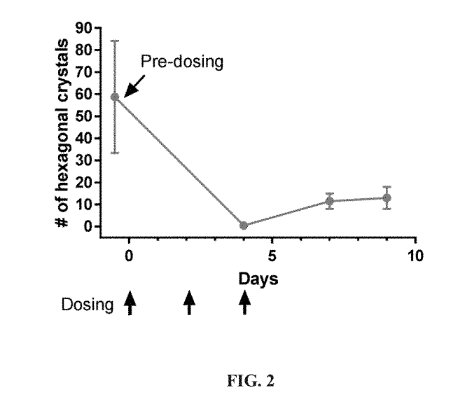

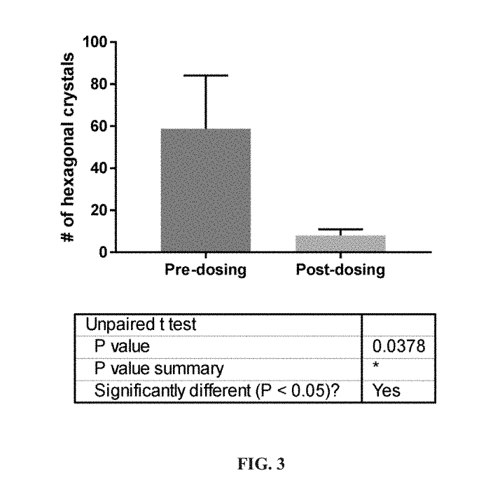

Two Scl3a1 male mice of C57BL/6N background of 16 and 9 weeks, respectively, received intraperitoneal administration of 50 mg/kg PEGylated hCGL-TV three times a week for 2 weeks. Animal studies are conducted in accordance with Aeglea BioTherapeutics, Inc. IACUC. Spot urine samples are collected prior to treatment start (day −11 and 0) and during treatment (day 4, 7, 9) to determine whether administration of hCGL-TV prevents or ameliorates cystine stone formation in the mice. Cystine crystals are assessed by bright field microscopy at 40× magnification and the number of crystals are counted from a representative region of interest consistent in each sample. FIGS. 2 and 3 graphically represent the number of hexagonal crystals visualized. The concentration of total cysteine in urine is measured by high performance liquid chromatography (HPLC) after reduction of total oxidized thiol content and protein precipitation, followed by derivation of total reduced thiol content with 7-Fluorobenzofurazan-4-sulfonic acid ammonium salt. Urinary creatinine is measured using the Creatinine Assay Kit obtained from Sigma-Aldrich (MAK079) and following the manufacturer's suggested procedure. Creatinine levels are used to normalize urinary total cysteine levels. FIG. 1 and Table 1 provide the total cysteine/creatinine levels over the course of the dosing schedule.

TABLE 1
Measurement of total cysteine and
creatinine in spot collection urine

| | Total cysteine (μM) | | Creatinine (mM) | | Mean of total |
|---|---|---|---|---|---|
| | Mouse | Mouse | Mouse | Mouse | cysteine/creatinine |
| Day | #1 | #2 | #1 | #2 | ratios (μM/mM) |
| −11 | 2622.393 | 4567.356 | 0.361 | 0.728 | 6769.044 |
| 0 | 3068.229 | 4922.726 | 0.450 | 0.791 | 6520.854 |
| 4 | 1805.064 | 1986.336 | 0.631 | 0.900 | 2533.840 |
| 7 | 3482.205 | 3286.022 | 0.585 | 0.577 | 5823.750 |
| 9 | 3762.806 | 4735.066 | 0.619 | 0.750 | 6196.134 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. No. 5,889,155
U.S. Pat. Publn. 2009/0304666
Austin-Ward and Villaseca, Revista Medica de Chile, 126(7):838-845, 1998.
Ausubel et al., Current Protocols in Molecular Biology, Greene Publishing Associates and Wiley Interscience, N.Y., 1994.
Bukowski et al., Clinical Cancer Res., 4(10):2337-2347, 1998.
Christodoulides et al., Microbiology, 144(Pt 11):3027-3037, 1998.
Davidson et al., J. Immunother., 21(5):389-398, 1998.
Doxsee et al., Sulfasalazine-induced cystine starvation: Potential use for prostate cancer therapy. The Prostate, 67(2):162-171, 2007.
Ercolani et al., Bladder outlet obstruction in male cystinuria mice. Int Urol Nephrol 42: 57-63, 2010.
Feliubadalo et al., Slc7a9-deficient mice develop cystinuria non-I and cystine urolithiasis. Hum Mol Genet, 12: 2097-2108, 2003.
Foye et al., Foye's Principles of Medicinal Chemistry, Lippincott Williams & Wilkins, 2007.
Gill and von Hippel, Calculation of protein extinction coefficients from amino acid sequence data. Anal Biochem, 182(2):319-326, 1989.
Glode et al., Cysteine auxotrophy of human leukemic lymphoblasts is associated with decreased amounts of intracellular cystathionase protein. Biochemistry, 20(5): 1306-1311, 1981.
Guan et al., The x c-cystine/glutamate antiporter as a potential therapeutic target for small-cell lung cancer: use of sulfasalazine. Cancer Chemotherapy and Pharmacology, 64(3):463-472, 2009.
Hanibuchi et al., Int. J. Cancer, 78(4):480-485, 1998.
Harkki et al., BioTechnology, 7:596-603, 1989.
Hellstrand et al., Acta Oncologica, 37(4):347-353, 1998.
Hollander, Front. Immun., 3:3, 2012.
Hopwood et al., In: Genetic Manipulation of Streptomyces, A Laboratory Manual, The John Innes Foundation, Norwich, Conn., 1985.
Hoover et al., The structure of human macrophage inflammatory protein-3alpha/CCL20. Linking antimicrobial and CC chemokine receptor-6-binding activities with human beta-defensins. J Biol Chem, 277(40):37647-37654, 2002.
Kim et al., Expression of cystathionine β-synthase is downregulated in hepatocellular carcinoma and associated with poor prognosis. Oncology Reports, 21(6):1449-1454, 2009.
Hui and Hashimoto, Infection Immun., 66(11):5329-5336, 1998.
Ito et al., J. Biochem., 79:1263, 1976.
Link et al., Cystathionase: a potential cytoplasmic marker of hematopoietic differentiation. Blut, 47(1):31-39, 1983.
Livrozet et al., An animal model of Type A Cystinuria due to spontaneous mutation in 129S2/SvPasCrl Mice. PLoS One 9:e102700, 2014.
Lordanescu, J. Bacteriol, 12:597 601, 1975.
Maniatis et al., Molecular Cloning, A Laboratory Manual, Cold Spring Harbor Press, Cold Spring Harbor, N.Y., 1988.
Mellor et al., Gene, 24:1-14, 1983.
Penttila et al., Gene, 61:155-164, 1987.
Peters et al., A mouse model for cystinuria type I. Hum Mol Genet 12: 2109-2120, 2003.
Qin et al., Proc. Natl. Acad. Sci. USA, 95(24):14411-14416, 1998.
Rao et al., Role of the Transsulfuration Pathway and of (gamma)-Cystathionase Activity in the Formation of Cysteine and Sulfate from Methionine in Rat Hepatocytes. Journal of Nutrition, 120(8):837, 1990.
Remington's Pharmaceutical Sciences, 18th Ed. Mack Printing Company, 1289-1329, 1990.
Schneider et al., 671 nih image to imageJ: 25 years of image analysis. Nature Methods, 9:2012.
Sibakov et al., Eur. J. Biochem., 145:567 572, 1984.
Stone et al., Strategies for optimizing the serum persistence of engineered human arginase I for cancer therapy. Journal of Controlled Release, 158:171-179, 2012.
Takakura et al., Assay method for antitumor L-methionine-lyase: comprehensive kinetic analysis of the complex reaction with L-methionine. Analytical Biochemistry, 327(2):233-240, 2004.
Tiziani et al., Optimized metabolite extraction from blood serum for 1H nuclear magnetic resonance spectroscopy. Analytical Biochemistry, 377:16-23, 2008.

Tiziani et al., Metabolomics of the tumor microenvironment in pediatric acute lymphoblastic leukemia. PLoS One, 8:e82859, 2013.

Ward, Proc, Embo-Alko Workshop on Molecular Biology of Filamentous Fungi, Helsinki, 119-128, 1989.
Wawrzynczak and Thorpe, In: Immunoconjugates, Antibody Comugates In Radioimaging And Therapy Of Cancer, Vogel (Ed.), NY, Oxford University Press, 28, 1987.
Zhang et al., Stromal control of cystine metabolism promotes cancer cell survival in chronic lymphocytic leukaemia. Nature Cell Biology, 14(3):276-286, 2012.
Zhao et al., Frequent Epigenetic Silencing of the Folate-Metabolising Gene Cystathionine-Beta-Synthase in Gastrointestinal Cancer. PLoS One, 7(11):e49683, 2012.

What is claimed is:

1. A method of treating a subject having or at risk of developing cystinuria comprising administering to the subject a therapeutically effective amount of a formulation comprising an isolated, modified primate cystathionine-γ-lyase (CGL) enzyme having at least two substitutions relative to a native primate CGL amino acid sequence (see SEQ ID NOs: 1 and 7-10), said at least two substitutions including a threonine at position 59 and a valine at position 339 of the native primate CGL sequence or a nucleic acid comprising a nucleotide sequence encoding the isolated, modified primate cystathionine-γ-lyase (CGL) enzyme.

2. The method of claim 1, wherein the enzyme further comprises a heterologous peptide segment.

3. The method of claim 2, wherein the heterologous peptide segment is an XTEN peptide, an IgG Fc, an albumin, or an albumin binding peptide.

4. The method of claim 1, wherein the enzyme is coupled to polyethylene glycol (PEG).

5. The method of claim 4, wherein the enzyme is coupled to PEG via one or more lysine or cystine residues.

6. The method of claim 1, wherein the subject is maintained on a L-cystine and/or L-cysteine restricted diet.

7. The method of claim 1, wherein the subject is maintained on a methionine-restricted diet.

8. The method of claim 1, wherein the subject is maintained on a normal diet.

9. The method of claim 1, wherein the subject is a human patient.

10. The method of claim 1, wherein the subject is a rodent.

11. The method of claim 1, wherein the formulation is administered intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctival, intravesicularlly, mucosally, intrapericardially, intraumbilically, orally, by inhalation, by injection, by infusion, by continuous infusion, by localized perfusion bathing target cells directly, via a catheter, or via a lavage.

12. The method of claim 1, wherein the subject has previously been treated for cystinuria and the enzyme is administered to prevent the recurrence of cystinuria.

13. The method of claim 1, further comprising administering at least a second cystinuria therapy to the subject.

14. The method of claim 13, wherein the second cystinuria therapy is a surgical therapy or a shock wave therapy.
